(19) United States
(12) Patent Application Publication
(10) Pub. No.: US 2012/0183585 A1
(43) Pub. Date: Jul. 19, 2012

(54) GEL BASED WOUND DRESSING AND A METHOD OF SYNTHESIZING THE SAME

(76) Inventors: Rassoul Dinarvand, Tehran (IR); Nasrin Samadi, Tehran (IR); Azade Taheri Borujeni, Tehran (IR)

(21) Appl. No.: 13/405,247

(22) Filed: Feb. 25, 2012

(51) Int. Cl. A61K 47/36; A61K 31/375; A61K 31/51; A61K 31/455; A61K 31/714; A61K 38/06; A61K 38/18; A61K 38/30; A61K 38/12; A61K 31/7036; A61K 31/18; A61K 38/16; A61K 9/14; A61P 43/00; A61P 31/00; A61P 39/06; A61P 3/02; A61K 47/38; B82Y 5/00; B82Y 40/00

(52) U.S. Cl. 424/400; 514/777; 514/781; 514/778; 514/772.5; 514/474; 514/276; 514/356; 514/52; 514/21.9; 514/8.2; 514/8.9; 514/8.5; 514/9.1; 514/2.3; 514/39; 514/603; 514/40; 977/773; 977/915; 977/900

(57) ABSTRACT

The various embodiments herein provide a gel based wound dressing comprising a lyophilized powder and a water-based solvent. The lyophilized powder comprises several nanoparticles and water miscible natural or synthetic polymers. The nanoparticles comprises pectin and a wound healing agent or an anti-microbial agent. The anti-microbial agent is nisin. The lyophilized powder and the water-based solvent are kept in two separate sealed packages and are mixed together before applying on a wound. The embodiments herein also provide a method of synthesizing the gel based wound dressing. The nano-particles control a release of the wound healing agent or the antimicrobial agent to a wound.

### BACKGROUND

1. Technical field

The embodiments herein generally relate to the wound dressings and particularly to the gel based wound dressings. The embodiments herein more particularly relate to the gel based wound dressings comprising nanoparticles that control the release of wound healing factors and antimicrobial agents. Moreover, the embodiments herein also relate to a method of synthesizing the gel based wound dressing having nanoparticles.

1. Description of the Related Art

Prior to 1960s, the wound dressings had a minimal role in the wound healing processes. In 1962, a concept of optimal environment for wound healing was developed by Winter. Later, many approaches were revolutionized in the wound care.

An ideal wound dressing should promote a rapid wound healing process. The wound dressings differ for different kinds of wounds. Not one specific dressing is suitable for all types of wounds. The differences in the cause, type, size and stage of wound healing of wounds have led to a vast array of wound dressings. The wound dressing should be sterile, protective, biocompatible, elastic, non-allergic, cost effective and comfortable for use. Also, the wound dressing should protect a wound from an infection and prevent a wound desiccation. Moreover, an ideal wound dressing should maintain optimum moisture content for a wound healing.

The wound dressings can be classified as either traditional or modern dressings. The traditional wound dressings include dry natural and synthetic bandages and gauzes. The traditional wound dressings do not provide optimum moisture content for the wound healing. The traditional wound dressings were replaced by the modern wound dressings for the chronic wounds and burns because the modern wound dressings provide a moist environment to facilitate a quick wound healing process. The gel wound dressing is an example of the modern gel wound dressing that is used for approximately all types of wounds i.e. dry and wet wounds. The gel wound dressings is easily removed from the wound, which leads to a high patient acceptability. Moreover, the gel wound dressings require moisture for an effective function. Apart from the development of improved wound dressings, an attention has been given to the possible use of wound healing and antimicrobial agents to promote the healing of wounds. An appropriate wound dressing containing wound healing or antimicrobial agents should control the release of these agents. The controlled delivery of wound healing and antimicrobial agents from the wound dressing prolongs the therapeutic effects of these therapeutic agents. Further, a controlled delivery of wound healing agents decreases the patient's exposure to excess amount of therapeutic agents and increases a patient compliance by reducing the frequency of changing the wound dressing. Several pharmaceutical modalities have been utilized in an attempt to improve the controlled release of wound healing and antimicrobial agents from the wound dressings. For example, nanotechnology has been used in development of novel wound dressings to control the release of wound healing and antimicrobial agents from the wound dressings. Moreover, the loading of wound healing agents in the nanoparticles protects them from degradation and accelerates the treatment of the wounds and reduces the cost of patient care significantly by reducing the necessary dose and the number of applications of these active agents. In addition, the nanoparticles containing the wound healing or antimicrobial agents penetrates into the deeper layers of a wound and a skin and then accelerate the wound healing process.

Hence there is a need to provide a gel based wound dressing containing nanoparticles for a controlled delivery of the wound healing and antimicrobial agents to a wound.

The above mentioned shortcomings, disadvantages and problems are addressed herein and which will be understood by reading and studying the following specification.

### OBJECTIVES OF THE EMBODIMENTS

The primary object of the embodiments herein is to provide a gel based wound dressing containing nanoparticles that control a delivery of the wound healing and antimicrobial agents to a wound.

Another object of the embodiments herein is to provide a gel based wound dressing that can be easily prepared.

Yet another object of the embodiments herein is to provide a gel based wound dressing that can be easily applied and easily removed from the wound.

Yet another object of the embodiments herein is to provide a gel based wound dressing that efficiently reduces an infection.

Yet another object of the embodiments herein is to provide a gel based wound dressing that is suitable for all types of wounds.

Yet another object of the embodiments herein is to provide a gel based wound dressing that is composed of biodegradable and water miscible polymers and therefore can be easily washed from the wound.

Yet another object of the embodiments herein is to provide a method of synthesizing the gel based wound dressing.

These and other objects and advantages of the embodiments herein will become readily apparent from the following detailed description taken in conjunction with the accompanying drawings.

### SUMMARY

The various embodiments herein provide a method and a composition for a gel based wound dressing. According to an embodiment herein, a gel based wound dressing comprises a lyophilized powder and a water-based solvent. The lyophilized powder includes a plurality of nanoparticles and water miscible natural or water miscible synthetic polymers. The plurality of nanoparticles includes a pectin and a wound healing agent or an anti-microbial agent. The water based solvent includes a purified water, a buffering agent and an isotonic agent. The lyophilized powder and the water-based solvent are kept in two separate sealed packages. The lyophilized powder and the water based solvent are mixed together before applying on a wound.

According to an embodiment herein, the water miscible natural or synthetic polymers are selected from a group consisting of pectin, chitosan, dextran, polyvinyl pirrolidone, and cellulose or starch derivatives. The cellulose or starch derivatives further include carboxymethyl cellulose, hydroxyethyl cellulose, hydroxypropyl cellulose, hydroxypropyl methyl cellulose, and carboxymethyl starch. The most preferred polymer used herein is pectin.

According to an embodiment herein, the wound healing agent includes any water soluble wound healing agent having an isoelectric pH of above 5. The wound healing agent mainly includes a vitamin, a growth factor and an antioxidant. The vitamin is selected from a group consisting of ascorbic acid (Vitamin C), thiamine (Vitamin B₁), niacin (Vitamin B₃), and cobalamin (Vitamin B₁₂). The growth factor is selected from a group consisting of a platelet derived growth factor (PDGF), a transforming growth factor (TGF-b), an insulin like growth factor (IGF), a fibroblast growth factor (FGF) and a Becaplermin. The antioxidant includes glutathione.

The antimicrobial agent includes an water soluble antimicrobial agent having an isoelectric pH of above 5. The antimicrobial agent is selected from a group consisting of bacitracin, polymyxin-b-sulfate, neomycin, mafenide, gentamycin and nisin. The preferred anti-microbial agent is nisin.

The plurality of nanoparticles controls a release of the wound healing agent or the antimicrobial agent to a wound. A ratio of the wound healing agent or the antimicrobial agent to the plurality of nanoparticles is 1:1 to 1:32 by weight. The wound healing agent or the antimicrobial agent is positively charged and the pectin is negatively charged. The plurality of nanoparticles has a mean diameter of 40-300 nm. The plurality of nanoparticles has a mean diameter which is less than about 250 nm. The plurality of nanoparticles has a mean diameter which is less than 200 nm. The plurality of nanoparticles is formed by an electrostatic interaction between the pectin and the wound healing agent or the anti-microbial agent. The plurality of nanoparticles have a zeta potential value equal to or less than −15 mV. The plurality of nanoparticles is present on the strands of the water miscible natural or water miscible synthetic polymers. The plurality of nanoparticles is joined by a charge by a charge bond or a hydrophilic-hydrophilic interaction or by a hydrophobic-hydrophobic interaction.

The embodiments herein also provide a method of synthesizing a gel based wound dressing. The method comprising the steps of first preparing a plurality of pectin nanoparticles containing a wound healing agent or an antimicrobial agent. The prepared plurality of pectin nanoparticles containing a wound healing agent or an antimicrobial agent is mixed with a mixture of water soluble polymers to form a gel. Then, the formed gel is lyophilized to form a lyophilized powder. A water based solvent is prepared separately. The water based solvent is prepared by mixing a purified water, a buffering agent and an isotonic agent. The prepared water based solvent is sterilized using a heat sterilizing method. The anti-microbial agent is nisin. The water soluble polymer is a pectin solution with a concentration of 4 mg/ml and a pH of 5.5. The gel is lyophilized at −40° C. for 48 h. The formed lyophilized powder and the prepared water based solvent are mixed together before an application over a wound to form the gel based wound dressing.

According to an embodiment herein, the step of preparing a plurality of pectin nanoparticles containing a wound healing agent or an antimicrobial agent further comprises preparing a solution of the pectin with a concentration of 1.25 mg/mL and a pH of 3.7-4.8. Further, a solution of a citric acid with a concentration of 0.2 mg/mL is prepared. The prepared solution of the pectin and the prepared solution of the citric acid are mixed with a predetermined amount of deionized water to form a mixture. The predetermined amount of the deionized water and an amount of the prepared solution of the citric acid is same and an amount of the prepared solution of the pectin is double the amount of the predetermined amount of deionized water and the amount of the prepared solution of the citric acid. Further, a solution of nisin is added to the formed mixture at a rate of 0.25 mL/min under a constant stirring at 1400 rpm to form dispersion. The concentration of the nisin solution is 0.2 mg/mL. The formed dispersion is kept under a constant stirring at 500 rpm for 10 min to obtain the plurality of pectin nanoparticles. The water based solvent includes an isotonic agent and a buffering agent. The isotonic agent is NaCl and the buffering agent is Dipotassium phosphate (K₂H₂PO₄). The amount of the pectin is more than an amount of the wound healing agent or the antimicrobial agent required for providing a protection. A ratio of the wound healing agent or the antimicrobial agent to the plurality of pectin nanoparticles is 1:1 to 1:32 by weight. The water miscible natural or water miscible synthetic polymers are selected from a group consisting of pectin, chitosan, dextran, polyvinyl pirrolidone, and cellulose or starch derivatives. The cellulose or starch derivatives include carboxymethyl cellulose, hydroxyethyl cellulose, hydroxypropyl cellulose, hydroxypropyl methyl cellulose, and carboxymethyl starch. The wound healing agent includes any water soluble wound healing agent having an isoelectric pH of above 5. The wound healing agent includes a vitamin, a growth factor and an antioxidant. The vitamin is selected from a group consisting of ascorbic acid (Vitamin C), thiamine (Vitamin B₁), niacin (Vitamin B₃), and cobalamin (Vitamin B₁₂). The growth factor is selected from a group consisting of a platelet derived growth factor (PDGF), a transforming growth factor (TGF-b), an insulin like growth factor (IGF), a fibroblast growth factor (FGF), Becaplermin. The antioxidant includes glutathione. The antimicrobial agent includes an water soluble antimicrobial agent having an isoelectric pH of above 5. The antimicrobial agent is selected from a group consisting of bacitracin, polymyxin-b-sulfate, neomycin, mafenide, gentamycin and nisin. Preferably, the anti-microbial agent is nisin. The plurality of nanoparticles controls a release of the wound healing agent or the antimicrobial agent to a wound. The wound healing agent or the antimicrobial agent is positively charged and the pectin is negatively charged. The plurality of nanoparticles has a mean diameter between 40-300 nm. The plurality of nanoparticles has a mean diameter of less than about 250 nm. The plurality of nanoparticles has a mean diameter of less than 200 nm. The plurality of nanoparticles has a zeta potential value equal to or less than −15 mV.

According to an embodiment herein, the gel wound dressing comprises nanoparticles that control the release of wound healing factors and antimicrobial agents for the treatment of wounds such as skin ulcers and burns.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 shows a flow chart illustrating the various steps involved in the synthesis of the gel based wound dressing, according to the embodiments herein.

FIG. 2 shows a flow chart illustrating the various steps involved in the preparation of plurality of pectin nanoparticles containing a wound healing agent or an antimicrobial agent i.e. the step described as 102 in FIG. 1, according to the embodiments herein.

FIG. 3 shows a flow chart illustrating a process for preparing a gel based wound dressing containing pectin nanoparticles, according to an embodiment herein.

FIG. 4 shows a schematic representation of steps of preparing the gel based wound dressing just before applying on a wound, according to an embodiment herein.

FIG. 5 shows a Dynamic Light Scattering (DLS) curve indicating a distribution of the size of nisin-pectin nanoparticles, according to the embodiments herein.

FIG. 6 shows a Differential Scanning Calorimetry (DSC) graphs of a pectin solution, a nisin solution and pectin-nisin nanoparticles, according to the embodiments herein.

FIG. 7 shows a curve indicating the effect of pH of pectin solution on the size and zeta potential of pectin-nisin nanoparticles, according to the embodiments herein.

FIG. 8 shows a curve indicating an effect of a pectin concentration on the size of pectin-nisin nanoparticles, according to the embodiments herein.

FIG. 9 shows a curve indicating an effect of a nisin solution concentration on the size of pectin-nisin nanoparticles, according to the embodiments herein.

FIG. 10A and FIG. 10B show the Scanning Electron Microscopic (SEM) images of nisin-pectin nanoparticles placed on pectin gel wound dressing with a magnification of 500 times and 15000 times respectively, according to the embodiments herein.

FIG. 11 shows a release profile of nisin from pectin gel wound dressing containing pectin-nisin nanoparticles, according to the embodiments herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient details to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a composition and a method for the preparation of a novel gel based wound dressing containing pectin nanoparticles. The pectin nanoparticles comprise the wound healing and the antibacterial agents that are useful for better treatment of wound. The novel wound dressing contains pectin nanoparticles. The pectin nanoparticles control a delivery of biologically active molecules such as antimicrobial agents and wound healing agents.

According to an embodiment herein, the method of synthesizing the gel based wound dressing comprises adding slowly the water soluble wound healing or antimicrobial agents with an isoelectric pH of above 5 to a pectin solution with a pH of 4.8 or less, under a constant stirring to form the pectin nanoparticles. The pectin nanoparticles containing the wound healing or antibacterial agents are prepared and extracted from the solution. Then the pectin nanoparticles are added to a solution of water miscible natural or synthetic polymers and mixed perfectly. The pectin nanoparticles get placed on the surface of the positive charged polymers by a charge-charge interaction or on the surface of other types of polymers by the hydrophilic-hydrophilic or the hydrophobic-hydrophobic interactions. Further, the prepared solution containing the water miscible natural or water miscible synthetic polymers and pectin nanoparticles are sterilized by a filtration method and lyophilized in the single-dose vials. A water based solvent comprising a buffering agent, an isotonic agents or a preservative are prepared in a separate container. The solvent is also sterilized using a heat sterilizing method. The lyophilized powder and the solvent are mixed just before a use. The easily poured gel prepared by mixing the lyophilized powder and the solvent are used on the wound directly. The nanoparticles control the release of the water soluble wound healing or antimicrobial agents and enhance the penetration of these agents to a deeper layer of wound.

According to an embodiment herein, a sterile-prepackaged gel wound dressing comprises a lyophilized powder and a water-based solvent. The sterile lyophilized powder and the water-based solvent are in two separate sealed packages. The sterile lyophilized powder having the water miscible natural or synthetic polymers forms a gel when mixed with a water-based solvent. The sterile water-based solvent contains purified water, the isotonic agents, the buffering agents or a preservative. The water soluble natural or water soluble synthetic polymers comprise the pectin nanoparticles on their surfaces. The pectin nanoparticles comprise a pectin and at least one bioactive compound such as a wound healing or an antimicrobial agent. The wound healing or antimicrobial agents are water soluble with an isoelectric pH of above 5. The pectin nanoparticles in the embodiments herein are stabilized by a charge-charge interaction between the pectin and the wound healing or antimicrobial agents. So, no cross linking agent is required in the embodiments herein.

In pectin nanoparticles, the ratio of the active agents to a pectin is about 1:1 to 1:32, by weight. The pectin nanoparticles have a mean diameter of the nanoparticles of about 100 to 200 nm. The pectin nanoparticles have a zeta potential of about −15 mV. The pectin nanoparticles are placed on the natural or synthetic polymers by ionic, hydrophilic-hydrophilic or hydrophobic-hydrophobic interactions. The lyophilized powder and the solvent are kept in two separately sealed packages. The lyophilized powder and the solvent are mixed just before the use in the lyophilized powder package. The lyophilized powder and the solvent form an easily poured gel formed by mixing the lyophilized powder and solvent. The easily poured gel is used to dress the wounds directly. The easily poured gel has a pH of 5.5-6.5. The pectin nanoparticles allow a controlled release of wound healing and antibacterial agents.

As used herein, the term “nanoparticles” refers to the particles with a mean diameter of 40-300 nm, typically the nanoparticles described in the embodiments herein have a mean diameter of less than about 250 nm and preferably less than 200 nm. The term “nanoparticles” herein also refers to the particles that are formed by an electrostatic interaction between pectin and the wound healing or antimicrobial agents. The nanoparticles according to the embodiments herein are stable and have a zeta potential value equal to or less than −15 mV.

Pectin is a complex carbohydrate found in the cell walls of the plants. Pectin is a water soluble and an ionic polysaccharide. When pectin is mixed with the wound healing or antimicrobial agents, under the conditions disclosed in the embodiments herein, the pectin nanoparticles with a mean size of 150 nm are formed without any additional cross-linking or stabilizing agents. These spontaneously formed electrostatic nanoparticles have an absolute value of zeta potential of more than −15 mV, and preferably more than −20 mV.

According to an embodiment herein, the gel wound dressing is directly used on the wound or the gel wound dressing is used in a hydrocolloid form. The gel-like characteristic of the gel wound dressing in the embodiments herein, facilitates in a removal of the dressing from a wound. An appropriate secondary wound dressing can also be preferably applied over the gel wound dressing. The secondary wound dressing adsorbs the exudes from the wounds and provides a physical protection on the wound. The gel wound dressing described in the embodiments herein is transparent which in turn helps to inspect the wound perfectly. The prepared gel containing pectin nanoparticles can be sterilized easily by filtration. The gel wound dressing can be pushed in a syringe and placed on the wound conveniently.

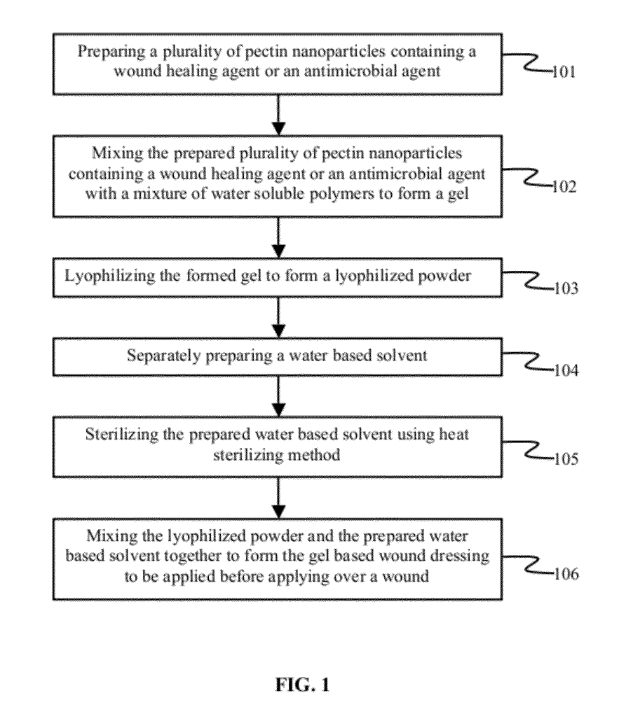

FIG. 1 shows a flow chart illustrating the steps involved in the synthesis of the gel based wound dressing, according to the embodiments herein. With respect to FIG. 1, a plurality of pectin nanoparticles containing a wound healing agent or an antimicrobial agent are first prepared (101). The anti-microbial agent is nisin. The prepared plurality of pectin nanoparticles containing a wound healing agent or an antimicrobial agent is mixed with a mixture of water soluble polymers to form a gel (102). The water soluble polymer is a pectin solution with a concentration of 4 mg/ml and a pH of 5.5. The formed gel is lyophilized to form a lyophilized powder (103). The solution is lyophilized at −40° C. for 48 h. Further, a water based solvent is prepared separately (104). The prepared water based solvent is sterilized using a heat sterilizing method (105). The heat sterilizing method is autoclave. The lyophilized powder and the prepared water based solvent is mixed together to form the gel based wound dressing to be applied before applying over a wound (106).

### Wound Healing Agent

According to the embodiments herein, the composition may also include an agent that functions as a wound healing agent. The wound healing agent refers to all the water soluble wound healing agents having their isoelectric pH above 5. The various examples of the wound heating agents include, but are not limited to, vitamins such as ascorbic acid (Vitamin C), thiamine (Vitamin B1), niacin (Vitamin B3), and cobalamin (Vitamin B12). Growth factors including platelet derived growth factor (PDGF), transforming growth factor (TGF-b), insulin like growth factor (IGF), fibroblast growth factor (FGF), and Becaplermin, and antioxidants including glutathione.

### Antimicrobial Agents

According to the embodiments herein, the antimicrobial agents refer to all the water soluble antimicrobial agents having their isoelectric pH above 5. The antimicrobial agents include, but are not limited to, bacitracin, polymyxin b sulfate, neomycin, mafenide, gentamycin and nisin. Nisin is a polycyclic antibacterial peptide with 34 amino acid residues used as a food preservative. The loading of wound healing agents in nanoparticles protects them from degradation and accelerates the treatment of wound and reduces the cost of patient care significantly by reducing the necessary dose and the number of applications. Nanoparticles containing the wound healing agents or the antimicrobial agents penetrate into a deeper layer of wound and skin and accelerate the wound healing process.

### Natural And Synthetic Polymers

According to the embodiments herein, the gel wound dressings refers to a wound dressing comprising the suitable materials including, but are not limited to, a natural and synthetic polymers. These polymers form a gel after coming in contact with the water based solvent, especially while it is applied to a wound. The mixture of water-miscible polymers includes, but is not limited to, pectin, chitosan, dextran, polyvinyl pirrolidone, and cellulose or starch derivatives. The starch derivatives include, for example, carboxymethyl cellulose, hydroxyethyl cellulose, hydroxypropyl cellulose, hydroxypropyl methyl cellulose, and carboxymethyl starch.

### Water-Based Solvent

The water-based solvent in the embodiments herein contains NaCl as isotonic agent and K₂H₂PO₄ as buffering agent. The water based solvent is made up of purified water. The water-based solvent is sterilized easily by means of heating under pressure i.e. autoclave. According to the amount of lyophilized powder, the amount of solvent can be determined to prepare an easily poured gel.

### Formation of Pectin Nanoparticles with Wound Healing Agents or Antimicrobial Agents

In the embodiments herein, the complexes of pectin and the wound healing or antimicrobial agents are preferably formed below the isoelectric point (pI) of the wound healing or antimicrobial agents and above the acid dissociation constant (pKa) of the pectin, wherein the wound healing agents or antimicrobial agents are positively charged and pectin is negatively charged. To obtain the stable pectin nanoparticles with a low aggregation, it is important to form the nanoparticles with a large net charge. Furthermore, one of the pectin or wound healing and antimicrobial agents has to be in excess. To provide a better protection to a wound, the amount of pectin should be in excess compared to the amount of wound healing and antimicrobial agents. To achieve this, the different ratios of wound healing or antimicrobial agents and pectin were studied in which the pectin particle size should be less than 200 nm and the zeta potential should be less than −15 mV.

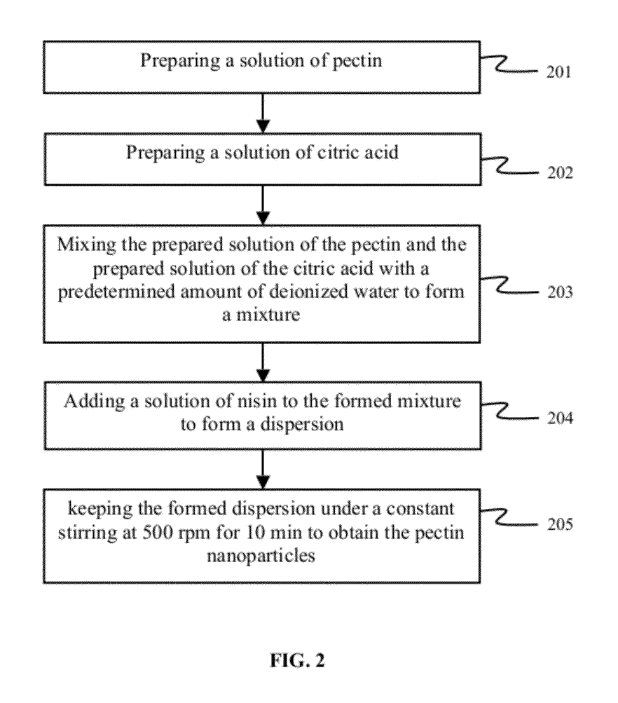

FIG. 2 shows a flow chart explaining the steps involved in the preparation of plurality of pectin nanoparticles containing a wound healing agent or an antimicrobial agent i.e. the step described as 102 in FIG. 1, according to the embodiments herein. With respect to FIG. 2, a solution of the pectin is prepared (201). The concentration of the pectin solution is 1.25 mg/ml and the pH of the pectin solution is 3.7-4.8. A solution of a citric acid is prepared (202). The concentration of citric acid solution is 0.2 mg/ml. The prepared solution of the pectin and the prepared solution of the citric acid are mixed with a predetermined amount of deionized water to form a mixture (203). A solution of nisin is added to the formed mixture (204). The solution of nisin is added in a concentration of 0.2 mg/ml at a rate of 0.25 ml/min under a constant stirring at 1400 rpm to form a dispersion. The dispersion is kept under a constant stirring at 500 rpm for 10 min to obtain the pectin nanoparticles (205).

### Experimental Data

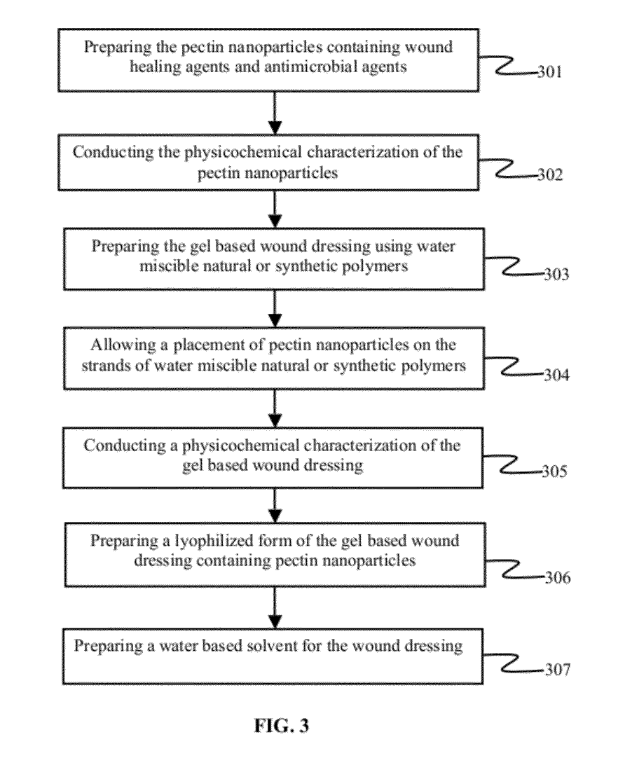

FIG. 3 shows a flow chart explaining the process for preparing a gel based wound dressing containing pectin nanoparticles, according to an embodiment herein. With respect to FIG. 3, pectin nanoparticles containing the wound healing agents and antimicrobial agents are prepared (301). Then, the physicochemical characterization of the pectin nanoparticles is conducted (302). The gel wound dressing is prepared using the water miscible natural or synthetic polymers (303). The pectin nanoparticles are allowed to get placed on the strands of water miscible natural or synthetic polymers (304). Then, the physicochemical characterization of the gel wound dressing is conducted (305). Then, the lyophilized form of gel wound dressing containing pectin nanoparticles is prepared (306). Then, a water based solvent is prepared separately (307). The water based solvent is prepared by mixing appropriate amounts of the isotonic agent and the buffering agent with purified water.

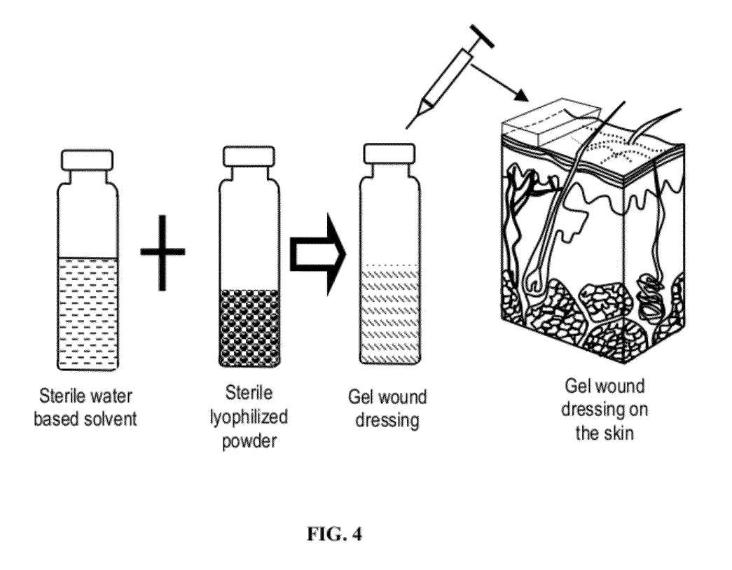

FIG. 4 shows a schematic representation of the steps of preparing the gel based wound dressing just before applying on a wound, according to an embodiment herein. With respect to FIG. 4, the sterile water based solvent is mixed with the sterile lyophilized powder to form the gel wound dressing. The gel wound dressing is applied on the wound using a syringe. The sterile water based solvent and the sterile lyophilized powder are kept in two separate sealed containers.

### EXAMPLE 1

Preparation of pectin, raisin and citric acid solutions: The gel based wound dressing was made from pectin and pectin nanoparticles that are placed on the surface of pectin strands. The Pectin solution with different concentrations, such as pectin solutions with concentrations of 1.25-5 mg/ml were freshly prepared in deionized water and stirred for complete hydration and then filtered using syringe a 0.45 micron filter (Millipore, USA). Nisin solutions with different concentrations of 0.1-40 mg/ml were prepared in deionized water and filtered using syringe 0.45 micron filter (Millipore, USA). The citric acid solutions with different concentrations of i.e. 0.2-1 mg/ml were freshly prepared in deionized water.

### EXAMPLE 2

Preparation of pectin-nisin nanoparticles: The pH of Pectin solution was adjusted to be within 3.7 to 4.8. In one sample, firstly the pH of Pectin solution was adjusted to be equal 3.7. Then, 1 ml of pectin solution (1.25 mg/ml), 0.5 ml of citric acid solution (0.2 mg/ml) and 0.5 ml deionized water were mixed in a container and were placed on a stirrer under 1400 rpm of constant stirring for 3 min. Pectin-nisin nanoparticles were prepared by addition of 1 ml of nisin solution (0.2 mg/ml) to the pectin and citric acid solution at rate of 0.25 ml/min under constant stirring (1400 rpm). After addition of nisin solution, the dispersion of pectin-nisin nanoparticles was kept on stirrer under constant stirring (500 rpm) for 10 min.

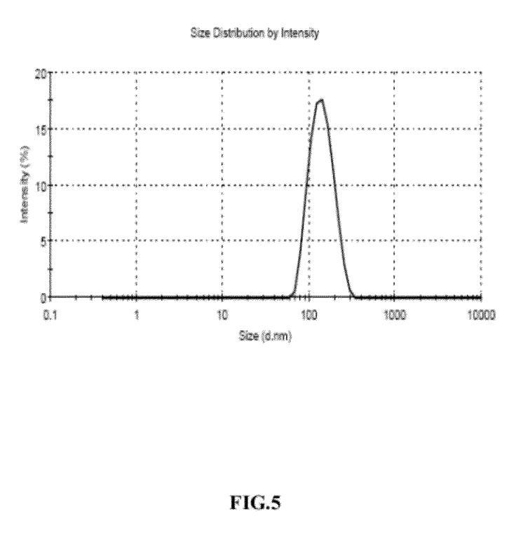

The size and zeta potential of pectin-nisin nanoparticles were analyzed using a Zetasizer nano ZS (Malvern instrument Ltd, United Kingdom). FIG. 5 shows a Dynamic Light Scattering (DLS) curve indicating distribution of the size of nisin-pectin nanoparticles with respect intensity, according to the embodiments herein. With respect to FIG. 5, the pectin-nisin nanoparticles had a size in the range of 100-200 nm and a zeta potential of about −15 mV.

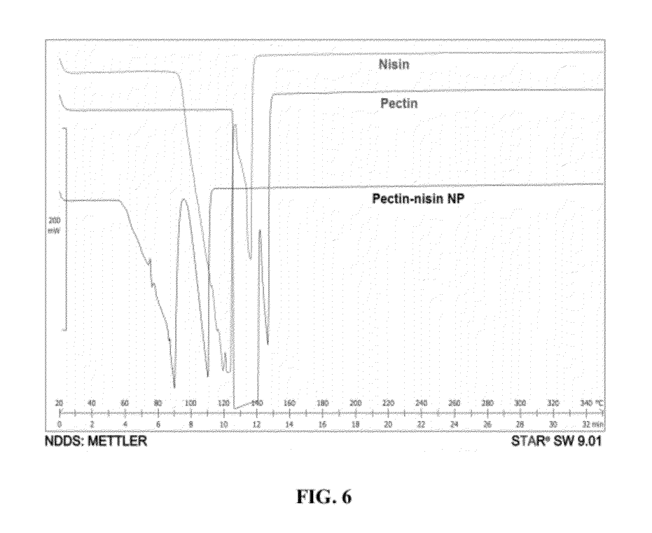

A differential scanning calorimetry (DSC) was used to verify the interaction between the pectin and the nisin in the pectin-nisin nanoparticles. The pectin solution (1.25 mg/ml), nisin solution (0.2 mg/ml) and pectin-nisin nanoparticles dispersion (nisin concentration: 0.2 mg/ml and pectin concentration: 0.75 mg/ml) were analyzed using DSC from 20° C. to 340° C., at a scan rate of 10° C/min. FIG. 6 shows a Differential Scanning calorimetry (DSC) graph/plot/curve of a pectin solution, a nisin solution and the pectin-nisin nanoparticles, according to the embodiments herein. With respect to FIG. 6, the interaction between the pectin and the nisin in the pectin-nisin nanoparticles was confirmed.

### EXAMPLE 3

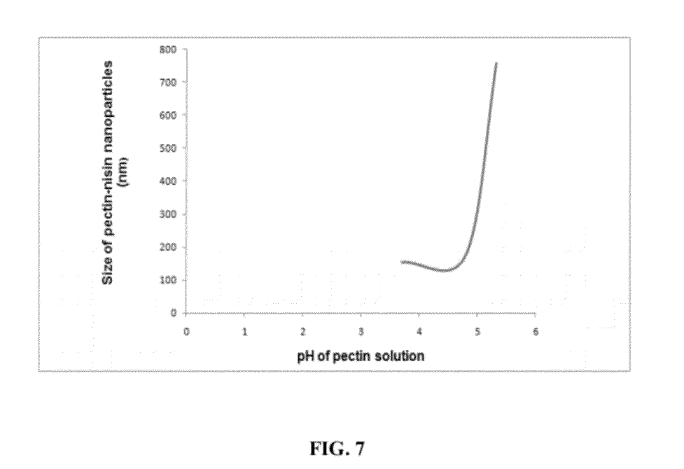

The effect of pH of pectin solution on the particle size and zeta potential of pectin-nisin nanoparticles: The effect of pH of pectin solution was evaluated on the particle size and zeta potential of pectin-nisin nanoparticles. A series of pectin solution (2.5 mg/ml) with different pH values ranging from 3.7 to 5.3 were prepared by using 0.1 N NaOH solution. Different pectin-nisin nanoparticles were prepared by mixing 1 ml of nisin solution (2.5 mg/ml), 0.5 ml of citric acid solution (0.2 mg/ml), 0.5 ml of deionized water and 1 ml of pectin solution (2.5 mg/ml) with different pH values in the range of 3.7-5.3. FIG. 7 shows a graphical representation showing the effect of pH of pectin solution on the size and zeta potential of pectin-nisin nanoparticles, according to the embodiments herein. As shown in FIG. 7, the increase in the pH of pectin solution above 4.8 results in an increase in the size of pectin-nisin nanoparticles significantly. Moreover, an increase of the pH of pectin solution decreases the zeta potential of pectin-nisin nanoparticles.

### EXAMPLE 4

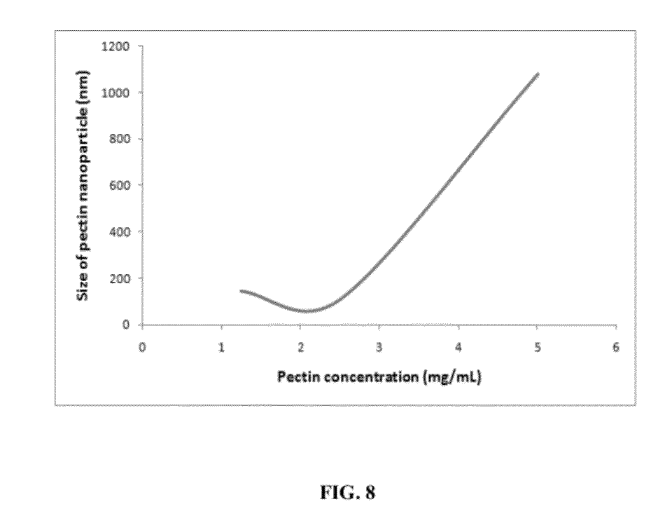

The effect of pectin concentration on the size and zeta potential of pectin-nisin nanoparticles was evaluated: The effect of pectin concentration on the size and zeta potential of pectin-nisin nanoparticles was evaluated. A series of pectin solution with different concentrations in the range of 1.25-5 mg/ml (pH 3.7) were prepared. Different pectin-nisin nanoparticles were prepared by mixing 1 ml of nisin solution (0.4 mg/ml), 0.5 ml of citric acid solution (0.2 mg/ml), 0.5 ml of deionized water and 1 ml of pectin solution with different concentrations (in the range of 1.25-5 mg/ml, pH 3.7). FIG. 8 shows a curve indicating the effect of the pectin concentration on the size of pectin-nisin nanoparticles, according to the embodiments herein. With respect to FIG. 8, an increase in the concentration of pectin solution above 2.5 mg/ml results in increasing the size of the pectin-nisin nanoparticles significantly. Moreover, an increase in the concentration of the pectin solution decreases the zeta potential of the pectin-nisin nanoparticles.

### EXAMPLE 4

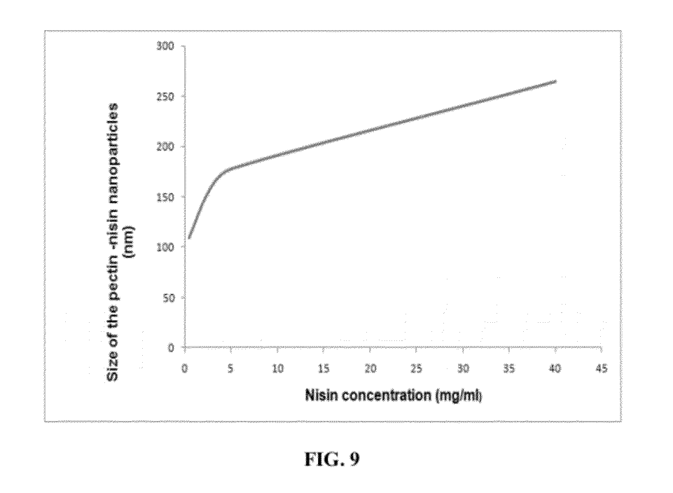

The effect of nisin concentration on the size and zeta potential of pectin-nisin nanoparticles: The effect of nisin concentration on the size and the zeta potential of the pectin-nisin nanoparticles was evaluated. A series of nisin solution with different concentrations in the range of 0.4-40 mg/ml were prepared. The pectin-nisin nanoparticles with different sizes were prepared by mixing 1 ml of pectin solution (2.5 mg/ml, pH 3.7), 0.5 ml of citric acid solution (0.2 mg/ml), 0.5 ml of deionized water and 1 ml of nisin solution with different concentrations (in the range of 0.4-40 mg/mL). FIG. 9 shows a curve indicating the effect of nisin solution concentration on the size of pectin-nisin nanoparticles, according to the embodiments herein. With respect to FIG. 9, an increase in the concentration of pectin solution above 5 mg/ml increases the size of pectin-nisin nanoparticles significantly. Moreover, an increase in the concentration of pectin solution increases the zeta potential of pectin-nisin nanoparticles.

### EXAMPLE 5

Preparation of pectin gel wound dressing containing pectin-nisin nanoparticles: The prepared pectin-nisin nanoparticles were placed in pectin solution with a concentration of 4 mg/ml and a pH of 5.5 and mixed sufficiently to form a gel. The gel was lyophilized at −40° C. for 48 h using a Lyotrap Plus lyophilizator (LTE, Scientific ltd, Oldham, UK).

The solvent of pectin gel containing the pectin-nisin nanoparticles was a water-based solvent that contained NaCl as isotonic agent and K₂H₂PO₄ as buffering agent. The solvent was sterilized using an autoclave.

### EXAMPLE 6

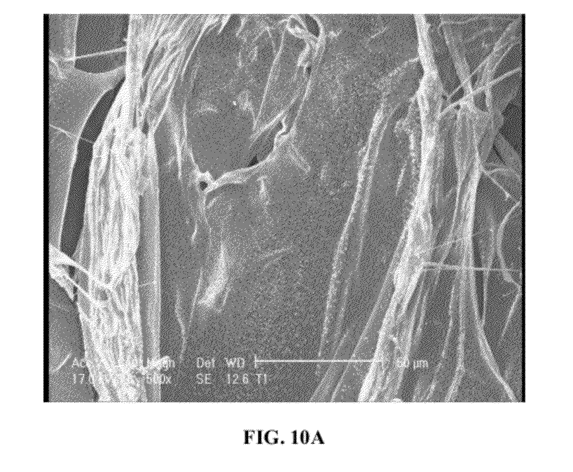

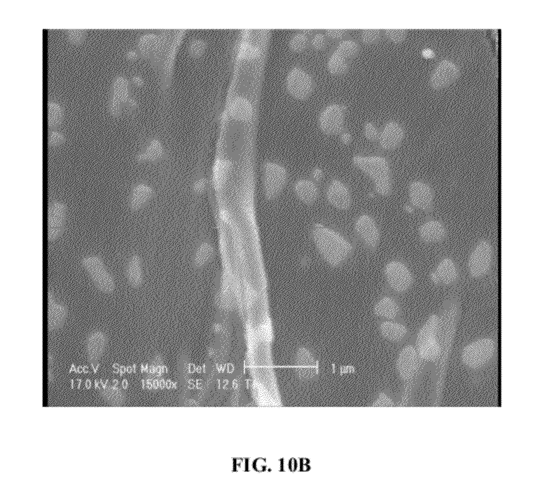

Scanning Electron Microscopy (SEM) analysis of pectin gel wound dressing containing pectin-nisin nanoparticles: After preparation of the pectin gel wound dressing containing the pectin-nisin nanoparticles, the prepared gel was analyzed using a Scanning Electron Microscope (SEM). FIG. 10A and FIG. 10B show the scanning electron microscopic images of nisin-pectin nanoparticles placed on pectin gel wound dressing under different magnifications, according to the embodiments herein. With respect to FIG. 10A and FIG. 10B, it was confirmed that pectin-nisin nanoparticles are successfully placed on the pectin gel wound dressing. Moreover, the size of particles was found to be between 100-300 nm.

### EXAMPLE 7

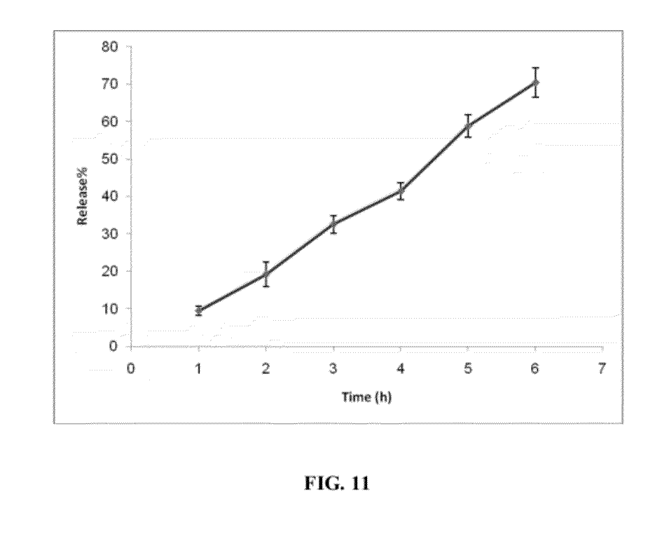

Release of nisin from a pectin gel wound dressing containing the pectin-nisin nanoparticles: The release of nisin from a pectin gel wound dressing containing the pectin-nisin nanoparticles was evaluated. For this purpose, 3 ml of pectin gel containing the pectin-nisin nanoparticles was placed in a dialysis tube (cellulose membrane, cutoff 8 kDa). The dialysis tube was placed in 10 ml PBS and the concentration of nisin was determined using Total Protein Kit, Micro Lowry, Peterson's Modification from Sigma-Aldrich. FIG. 11 shows a release profile of a nisin from a pectin gel wound dressing containing the pectin-nisin nanoparticles, according to the embodiments herein. With respect to FIG. 11, it is clear that pectin gel wound dressing containing the pectin-nisin nanoparticles can provide a controlled release of a nisin during a longer period of time. The release of nisin had a zero-order kinetic and there was no burst release in the release profile of nisin from pectin gel wound dressing containing pectin-nisin nanoparticles.

### EXAMPLE 8

Antibacterial properties of a gel wound dressing containing the pectin-nisin nanoparticles: To evaluate the antibacterial properties of a gel wound dressing containing the pectin-nisin nanoparticles, the micro-organisms such as, Escherichia coli and Pseudomonas aeruginosa were used as representative gram-negative bacteria and Staphylococcus aureus was used as a gram-positive bacterium.

Minimum inhibitory concentrations (MIC): The MICs of a gel wound dressing containing the pectin-nisin nanoparticles and a mixture of nisin, pectin and citric acid solutions were determined by a micro-dilution method by using 96 U-shaped wells plates. The samples were diluted in each row of the microtiter plate from 1:2 to 1:128 by mixing 100 μl of each sample with 100 μl of Mueller-Hinton broth (MHB). The concentration of nisin in the first tube and the last tube was 0.1 and 0.0015 mg/ml, respectively. The stock microbial suspensions (1×10⁶ CFU ml⁻¹) were prepared from 24 h old cultures in MHB. Then test strain inoculums were added to each well to reach the final inoculum size of about 5×10⁵ CFU ml⁻¹. After 24 h incubation at 35° C., the microtiter plates were tested for the absence or presence of a visible growth. The endpoint MIC is the lowest concentration of the compound at which the test strain does not demonstrate visible growth.

The MIC of a pectin gel wound dressing containing the pectin-nisin against Staphylococcus aureus, Escherichia coli and Pseudomonas aeruginosa were 16, 8 and 8 folds lower than the MIC of the mixture of nisin, pectin and citric acid solutions, respectively. For the determination of inhibition zone of a pectin gel wound dressing containing the pectin-nisin and the mixture of nisin, pectin and citric acid solutions, Mueller-Hinton agar plates were inoculated individually with Escherichia coli, Pseudomonas aeruginosa and Staphylococcus aureus. Then wells were done by punching a stainless steel cylinder onto the agar plates and removing the agar to form a well. A 100 μl aliquot of each sample was placed individually in three wells. The plates were incubated at 35° C. for 24 h. After an incubation, the mean inhibition zone diameter around each well was determined in millimeter. Each assay was carried out in triplicate.

The results show that the inhibition zones of a gel wound dressing containing the pectin-nisin nanoparticles against Staphylococcus aureus, Escherichia coli and Pseudomonas aeruginosa were 11, 8 and 9 mm greater than inhibition zone of the mixture of nisin, pectin and citric acid solutions, respectively.

Hence, the embodiments herein provide a wound dressing for a controlled release of the biologically active wound healing agents and the antimicrobial agents to the wounds. The sterile-pre-packaged gel based wound dressing system disclosed in the embodiments herein includes a lyophilized powder and a water-based solvent. The lyophilized powder and the solvent are stored in two separate sealed packages and are mixed just before a use. The easily poured gel is formed by mixing the lyophilized powder and the aqueous solvent and is applied to dress the wounds directly. The lyophilized powder is composed of water miscible natural or synthetic polymers and the pectin nanoparticles. The pectin nanoparticles are placed on the strands of water miscible natural or synthetic polymers by charge-by-charge bonds or hydrophilic-hydrophilic or hydrophobic-hydrophobic interactions. The pectin nanoparticles contain both the wound healing agents and the antibacterial agents. The pectin nanoparticles allow the controlled release of the wound healing and the antibacterial agents. The easy preparation, easy application and removal, and the efficient reduction of the infection rate makes the gel based wound dressing, a suitable wound dressing for all types of wounds. In addition to the above, the gel based wound dressing is made up of biodegradable and water miscible polymers and therefore can be easily washed from the wound.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

what is claimed is:

1. A gel based wound dressing comprises:
a lyophilized powder, wherein the lyophilized powder includes a plurality of nanoparticles and a water miscible natural or a water miscible synthetic polymer, and wherein the plurality of nanoparticles include pectin and a wound healing agent or an anti-microbial agent; and
a water-based solvent, wherein the water based solvent includes a buffering agent and an isotonic agent, and wherein the lyophilized powder and the water-based solvent are kept in two separate sealed packages and wherein the lyophilized powder and the water based solvent are mixed together before applying on a wound.

2. The wound dressing according to claim 1, wherein the water miscible natural or the water miscible synthetic polymers are selected from a group consisting of pectin, chitosan, dextran, polyvinyl pirrolidone, cellulose or starch derivatives, and wherein the cellulose or starch derivatives include carboxymethyl cellulose, hydroxyethyl cellulose, hydroxypropyl cellulose, hydroxypropyl methyl cellulose, and carboxymethyl starch, and wherein the polymer is pectin.

3. The wound dressing according to claim 1, wherein the wound healing agent includes any water soluble wound healing agent having an isoelectric pH above 5, and wherein the wound healing agent includes a vitamin, a growth factor and an antioxidant, and wherein the vitamin is selected from a group consisting of ascorbic acid (Vitamin C), thiamine (Vitamin B₁), niacin (Vitamin B₃), cobalamin (Vitamin B₁₂), and wherein the growth factor is selected from a group consisting of a platelet derived growth factor (PDGF), a transforming growth factor (TGF-b), an insulin like growth factor (IGF), a fibroblast growth factor (FGF), Becaplermin, and wherein the antioxidant includes glutathione.

4. The wound dressing according to claim 1, wherein the antimicrobial agent includes any water soluble antimicrobial agent having an isoelectric pH value above 5, and wherein the antimicrobial agent is selected from a group consisting of bacitracin, polymyxin-b-sulfate, neomycin, mafenide, gentamycin and nisin, and wherein the anti-microbial agent is nisin.

5. The wound dressing according to claim 1, wherein the plurality of nanoparticles control a release of the wound healing agent or the antimicrobial agent to a wound, and wherein a ratio of the wound healing agent or the antimicrobial agent to the plurality of nanoparticles is 1:1 to 1:32 by weight.

6. The wound dressing according to claim 1, wherein the wound healing agent or the antimicrobial agent is positively charged and wherein the pectin is negatively charged.

7. The wound dressing according to claim 1, wherein the plurality of nanoparticles have a mean diameter of 40-300 nm, and wherein the plurality of nanoparticles have a mean diameter less than about 250 nm, and wherein the plurality of nanoparticles have a mean diameter less than 200 nm.

8. The wound dressing according to claim 1, wherein the plurality of nanoparticles are formed by an electrostatic interaction between the pectin and the wound healing agent or the anti-microbial agent.

9. The wound dressing according to claim 1, wherein the plurality of nanoparticles have a zeta potential value equal to or less than −15 mV.

10. The wound dressing according to claim 1, wherein the plurality of nanoparticles are present on strands of the water miscible natural or the water miscible synthetic polymers and wherein the plurality of nanoparticles are joined by a charge-charge bond or a hydrophilic-hydrophilic interaction or by a hydrophobic-hydrophobic interaction.

11. A method of synthesizing a gel based wound dressing comprising the steps of:
preparing a plurality of pectin nanoparticles containing a wound healing agent or an antimicrobial agent, and wherein the anti-microbial agent is nisin;
mixing the prepared plurality of pectin nanoparticles containing the wound healing agent or the antimicrobial agent with a mixture of water soluble polymers to form a gel, and wherein the water soluble polymer is a pectin solution with a concentration of 4 mg/ml and a pH of 5.5;
lyophilizing the formed gel to form a lyophilized powder, wherein the gel is lyophilized at −40° C. for 48 h; and
separately preparing a water based solvent, wherein the water based solvent is prepared by mixing a buffering agent and an isotonic agent;
sterilizing the prepared water based solvent using a heat sterilizing method, and wherein the formed lyophilized powder and the prepared water based solvent are mixed together before an application over a wound to form the gel based wound dressing.

12. The method according to claim 11, wherein the step of preparing the plurality of the pectin nanoparticles containing the wound healing agent or the antimicrobial agent further comprises:
preparing a solution of the pectin with a concentration of 1.25 mg/ml, wherein a pH of the pectin solution is 3.7-4.8;
preparing a solution of a citric acid with a concentration of 0.2 mg/ml;
mixing the prepared solution of the pectin and the prepared solution of the citric acid with a predetermined amount of deionized water to form a mixture, wherein the predetermined amount of the deionized water and an amount of the prepared solution of the citric acid is same, and wherein an amount of the prepared solution of the pectin is double the amount of the predetermined amount of deionized water and the amount of the prepared solution of the citric acid;
adding a solution of a nisin to the formed mixture at a rate of 0.25 ml/min under a constant stirring at 1400 rpm to form a dispersion, and wherein a concentration of the solution of the nisin is 0.2 mg/ml;
keeping the formed dispersion under a constant stirring at 500 rpm for 10 min to obtain the plurality of pectin nanoparticles.

13. The method according to claim 11, wherein the isotonic agent is NaCl and wherein the buffering agent is Dipotassium phosphate (K₂H₂PO₄).

14. The method according to claim 11, wherein an amount of pectin is more than an amount of the wound healing agent or the antimicrobial agent required for providing a protection, and wherein a ratio of the wound healing agent or the antimicrobial agent to the plurality of pectin nanoparticles is 1:1 to 1:32 by weight.

15. The method according to claim 11, wherein the water soluble polymers further includes chitosan, dextran, polyvinyl pirrolidone, cellulose or starch derivatives, and wherein the cellulose or starch derivatives include carboxymethyl cellulose, hydroxyethyl cellulose, hydroxypropyl cellulose, hydroxypropyl methyl cellulose, and carboxymethyl starch.

16. The method according to claim 11, wherein the wound healing agent includes any water soluble wound healing agent having an isoelectric pH above 5, and wherein the wound healing agent includes a vitamin, a growth factor and an antioxidant, and wherein the vitamin is selected from a group consisting of ascorbic acid (Vitamin C), thiamine (Vitamin B₁), niacin (Vitamin B₃), cobalamin (Vitamin B₁₂), and wherein the growth factor is selected from a group consisting of platelet derived growth factor (PDGF), transforming growth factor (TGF-b), insulin like growth factor (IGF), fibroblast growth factor (FGF), Becaplermin, and wherein the antioxidant includes glutathione.

17. The method according to claim 11, wherein the antimicrobial agent includes any water soluble antimicrobial agent having an isoelectric pH above 5, and wherein the antimicrobial agent is selected from a group consisting of bacitracin, polymyxin-b-sulfate, neomycin, mafenide, gentamycin and nisin, and wherein the anti-microbial agent is nisin.

18. The method according to claim 11, wherein the plurality of pectin nanoparticles control a release of the wound healing agent or the antimicrobial agent to a wound.

19. The method according to claim 11, wherein the wound healing agent or the antimicrobial agent is positively charged and wherein the pectin is negatively charged.

20. The method according to claim 11, wherein the plurality of pectin nanoparticles have a mean diameter between 40-300 nm, and wherein the plurality of nanoparticles have a mean diameter less than 250 nm, and wherein the plurality of nanoparticles have a mean diameter less than 200 nm, and wherein the plurality of nanoparticles have a zeta potential value equal to or less than −15 mV.
